(19) United States
(12) Patent Application Publication
(10) Pub. No.: US 2004/0176704 A1
(43) Pub. Date: Sep. 9, 2004

(54) Collection device adapted to accept cartridge for point of care system

(76) Inventors: Timothy A. Stevens, Warwick, NJ (US); C. Mark Newby, Tuxedo, NY (US); Bradley M. Wilkinson, North Haledon, NJ (US); Robert G. Ellis, Wykoff, NJ (US); Charles G. Hwang, Ridgewood, NJ (US); Frank Francavilla, Newton, NJ (US); Jamieson W. Crawford, New York, NY (US)

Correspondence Address: Scott J. Rittman, Esq., Becton, Dickinson and Company, 1 Becton Drive, Franklin Lakes, NJ, 07417-1880, US

(21) Appl. No.: 10/377,620

(22) Filed: Mar. 4, 2003

(51) Int. Cl. A61B 5/00

(52) U.S. Cl. 600/584

(57) ABSTRACT

A system is provided which may be used for collecting and testing fluid samples at a patient point of care location. A disposable cartridge and collection device is provided for collecting a fluid sample directly into a cartridge containing testing mechanisms for immediate evaluation of the collected sample. The cartridge contains an array of electrical contacts, electrochemical sensors and circuitry configured to electrically couple with a hand-held analytical device, such as a personal digital assistant (PDA) or a stand-alone computer workstation, which controls the testing of the fluid sample within the cartridge and provides a rapid indication of test results at the point of care.

### CROSS-REFERENCE TO RELATED APPLICATIONS

Related subject matter is disclosed in a U.S. Patent Application of Stevens et al. entitled “Cartridge Having An Integrated Collection Element For Point Of Care System”, Attorney Docket No. 43699, filed concurrently and the entire contents of which being incorporated herein by reference.

### FIELD OF THE INVENTION

The present invention relates to a system for capturing and testing fluid samples at a patient point of care location. In particular, the present invention relates to a system for collecting a fluid sample such as blood, directly into a sealed cartridge containing testing mechanisms for evaluation of the collected sample immediately upon collection. The cartridge contains an array of electrical contacts, electrochemical sensors (i.e., biosensor chips) and circuitry configured to electrically couple with a remote analytical device, such as a hand-held personal digital assistant (PDA), or a stand-alone computer workstation, which controls testing of the fluid sample within the cartridge and provides a rapid indication of test results at the point of care.

### BACKGROUND OF THE INVENTION

In a typical healthcare environment, clinical laboratories often perform numerous tests for doctors and healthcare professionals. Such laboratories perform these tests on various fluid samples, such as human blood, urine, plasma, serum or other fluids in order to measure chemical or physical properties of the samples. The results of these tests are used by doctors and healthcare professionals to make clinical decisions related to patient care and treatment. Because such results are used to make decisions for patient care, dependable test results are of the utmost importance. However, in addition to dependability considerations, many situations may require immediate determination of test results for effective care and treatment. In such cases, remote laboratory facilities are often unable to provide test results in a useful manner. As pointed out in U.S. Pat. No. 5,096,669 issued to Lauks et al., the entire content of which is incorporated herein by reference, many situations require test results immediately, such as in the physicians office, hospital emergency room or at the patient's bedside.

In any sample testing scenario, the first consideration typically concerns sample collection and thereafter, sample transfer to a testing facility or apparatus. As discussed in U.S. Pat. No. 6,074,383 issued to Grippi et al., the entire content of which is incorporated herein by reference, the taking of samples such as blood, is considered a necessary part of the process of diagnosing and controlling many forms of disease. As described in the Grippi patent, blood samples are obtained by puncturing the skin of a patient's finger with a sharp object such as a syringe or pointed blade, which are typically disposable, such that once used, each may be discarded. Details of syringe construction and use in sample collection are described in U.S. Pat. No. 6,196,998 issued to Jansen et al., the entire content of which is incorporated herein by reference. Such conventional syringes described in the Jansen Patent include a barrel having an open distal end, typically engaged to a needle assembly with a needle cannula, and an opposed proximal end with a cylindrical wall extending between ends and defining a substance retaining chamber. As may be appreciated by those skilled in the art, collection of a sample within the retaining chamber of the syringe merely requires needle insertion at the distal end, and a sliding movement of a plunger within the chamber from the proximal end.

One alternative to the syringe as a blood sample collection device is discussed in the Grippi patent referenced above. Lasers, commonly known as laser lancets, may be used as a substitute for a needle or pointed blade for obtaining blood samples from patients. A laser lancet, as with a mechanical lancet, can be used to puncture the surface of the skin for exposing blood samples where the blood may then be collected for analysis.

As pointed out in U.S. Pat. No. 6,221,307 issued to Norman J. Hutton, the entire content of which is incorporated herein by reference, the collected blood samples may be taken and analyzed in hospital or clinical situations for various medical purposes. Sample analysis may include detection of pH, pCO₂, pO₂, Na⁺, Ca⁺⁺, K⁺, hematocrit and glucose levels in the sample, in addition to sample temperature measurements through the use of real time sensors such as those described in U.S. Pat. No. 5,212,050 issued to Mier et al., and in U.S. Pat. No. 5,200,051 issued to Cozzette et al., the entire content of each being incorporated herein by reference.

Collection, handling and testing of these samples typically requires the use of various medical testing instruments and, as pointed out in the Hutton patent referenced above, collection ideally occurs using standard sized collection devices. The use of standard sized collection devices allows the design and use of testing instruments which are configured to process samples without removal from the collection device. One such form of testing instrument currently available is a hand-held analyzer, which may be configured to accept samples contained within a standard collection device. Hand-held analyzers for sample testing are extensively discussed in U.S. Pat. No. 6,066,243 issued to Anderson et al., and in U.S. patent application Ser. No. 2002/0002326 issued to Causey et al., the entire content of each being incorporated herein by reference. Many such analyzers are configured to accept samples for testing via access ports adapted to receive small containment cartridges containing the sample for evaluation. Analyzers such as PDA-based devices are very cost effective, easily upgraded and allow on the spot analysis. Additional details regarding such configurations are discussed in U.S. Pat. No. 5,096,669, referenced above, in an article by Jason Thibeault entitled “Move Toward PDA-Based Devices Gets Boost from FDA”, Medical Device & Diagnostic Industry, August 2002, in an article by Ian Austin entitled “Palmtops In The Operating Room”, New York Times, Aug. 22, 2002, and in an article by Stephanie De Ritis entitled “Expanding Exceeding POCT Boundaries”, Advance/Laboratory, August 2002, the entire content of each being incorporated herein by reference.

The containment cartridge method of sample collection and testing has proved successful in many applications. Containment cartridges include a small containment chamber into which a fluid sample is placed for testing, typically via a capillary tube placed into contact with an exposed fluid sample source. The chamber includes an extensive sensor array, such that numerous tests and evaluations may be performed on the contained sample. The cartridge is built as a standardized package which is configured to fit within an access port on a testing device that electrically couples to the sensor array of the cartridge, and directly collects information from the sensors regarding the contained sample. The move to standardize devices for interfacing between workstations and clinical systems is discussed further in the article entitled “Expanding Exceeding POCT Boundaries”, referenced above. One such containment cartridge compatible with a hand-held analyzer is discussed in U.S. Pat. No. 5,638,828 issued to Lauks et al., the entire content of which is incorporated herein by reference, and in U.S. Pat. No. 5,096,669 also issued to Lauks et al. and referenced above. The Lauks patents disclose a collection device for collecting a volume of blood or other fluids in a capillary tube housed within a sealed cartridge for diagnostic testing using a hand-held analytical device. As discussed in the Lauks patents, a fluid sample is introduced into a disposable cartridge through an orifice at one end of the cartridge. The sample enters the cartridge by putting the orifice in contact with an exposed source and a sample is drawn by capillary action into a conduit within the cartridge.

However, the cartridge disclosed in the Lauks patents requires that a medical professional first prick the patient's finger with a finger stick to draw a small amount of blood through a dermal puncture. The medical professional is then required to place the orifice of the cartridge in contact with the blood sample formed on the dermal puncture in the patient's finger to draw the blood into the conduit of the collection cartridge. This method requires an exposed fluid sample f6r collection by the collection cartridge. An alternative collection method is also described in the Lauks patents wherein a syringe device is used to collect a sample then transfer the sample to the orifice of the cartridge or a reservoir chamber within the cartridge. This method requires additional steps to transfer the collected sample from a syringe to the cartridge which, depending on cartridge size and construction, may be difficult, time consuming and prone to contamination. In addition, as pointed out in the Lauks patents, transfer of exposed blood samples includes the risk of spills, contamination and transmission of infectious diseases such as human immunodeficiency virus or hepatitis.

Therefore, a need exists to provide a cartridge assembly for directly collecting, containing and testing fluid samples such as blood, in association with hand-held analytical devices or stand-alone computer workstations, without requiring exposed sample sources or difficult sample transfers from a collection device to a cartridge reservoir.

### SUMMARY OF THE INVENTION

An object of the present invention is to provide a cartridge assembly and a sample collection device, such as a syringe, which may be used in combination for directly collecting, containing and testing fluid samples such as blood, without sample transfers or sample exposure.

This and other objects are substantially achieved by providing a system for collecting a fluid sample directly into a sealed cartridge containing testing mechanisms for evaluation of the collected sample immediately upon collection. The cartridge is removably connected to a sample collection device such as a syringe, and collects a fluid sample for testing from the collection device apparatus. Once a sample has been collected, the cartridge may be removed from the collection device and engaged with a remote analytical testing device. Where the collection device allows shielding or retraction of the collection needle, lancet or other piercing element, the cartridge may remain safely engaged with the collection device during testing. The cartridge contains an array of electrical contacts, electrochemical sensors and circuitry configured to electrically couple with a remote analytical device such as a hand-held personal digital assistant (PDA), which controls the testing of the fluid sample within the cartridge and provides a rapid indication of test results at the point of care.

### BRIEF DESCRIPTION OF THE DRAWINGS

These and other objects, advantages and novel features of the invention will be more readily appreciated from the following detailed description when read in conjunction with the accompanying drawings, in which:

FIG. 1 is a view of an example of a removable cartridge system in use with a collection device according to an embodiment of the present invention;

FIG. 2 is a view of the removable cartridge of FIG. 1 engaged with a collection device according to an embodiment of the present invention;

FIG. 3 is a view of the removable cartridge of FIG. 1 engaged with a hand-held analytical device according to an embodiment of the present invention;

FIG. 4 is a view of an example of a removable cartridge system in use with a lancet collection device according to an embodiment of the present invention;

FIG. 5 is a view of the removable cartridge of FIG. 4 according to an embodiment of the present invention;

FIG. 6 is a view of the removable cartridge of FIG. 4 engaged with a collection device according to an embodiment of the present invention;

FIG. 7 is a view of an example of an evacuated removable cartridge system in use with a collection device according to an embodiment of the present invention;

FIG. 8A is a view of the distal end of the evacuated removable cartridge of FIG. 7 according to an embodiment of the present invention;

FIG. 8B is a view of the proximal end of the evacuated removable cartridge of FIG. 7 according to an embodiment of the present invention and

FIG. 9 is a view of the evacuated removable cartridge of FIG. 7 engaged with a collection device according to an embodiment of the present invention.

In the drawing figures, it will be understood that like numerals refer to like structures.

### DETAILED DESCRIPTION OF THE PREFERRED EMBODIMENTS

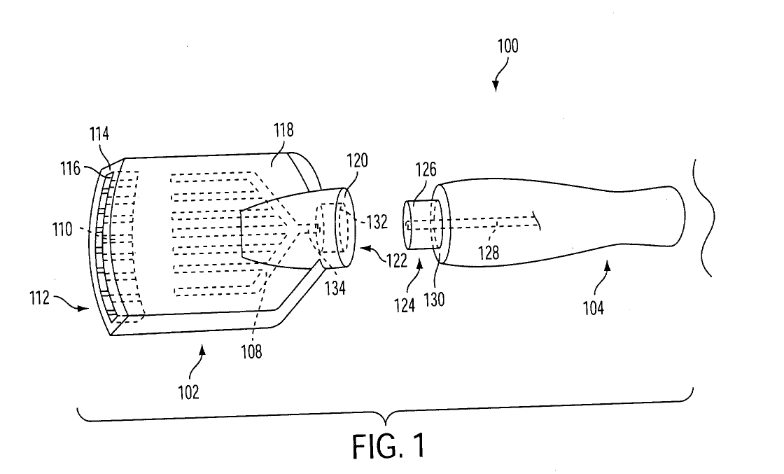

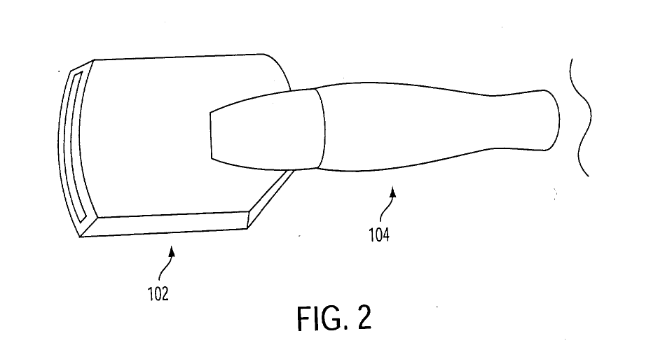

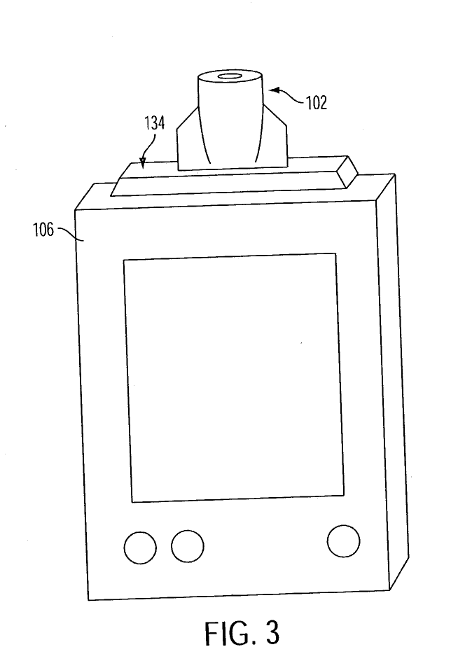

A diagram of an exemplary system 100 in accordance with an embodiment of the present invention is shown in FIG. 1. For the following discussion, reference will be made to FIGS. 1, 2 and 3, and as necessary, attention will be drawn to a particular figure. FIG. 1 is a view in partial section of a collection cartridge 102 disengaged from a collection device 104. FIG. 2 is a view of the removable cartridge 102 engaged with the collection device 104 of FIG. 1, and FIG. 3 is a view of the removable cartridge 102 of FIG. 1 engaged with a hand-held analytical device 106, each according to an embodiment of the present invention.

The system 100 of FIG. 1 preferably includes a disposable cartridge 102 for providing a containment chamber 108 into which fluid samples are placed by a collection device for analysis. As known to those skilled in the art, a collection device 104 such as a syringe or lancet, may be used to safely collect a fluid sample into a device body cavity or passageway. The cartridge 102 is adapted to removably engage the collection device 104 and allow the collection device to collect a fluid sample directly into the containment chamber 108 of the cartridge 102. Once a sample is collected into the cartridge 102, the cartridge may be removed from the collection device 104 and engaged with a remote analytical device 106 for testing. Where the collection device 104 allows shielding or retraction of the collection element, such as a needle or lancet, the cartridge 102 may remain safely engaged with the collection device during testing. It will be noted that the embodiments below include needle and lancet assemblies, however, the apparatus of the present invention works just as well with other collection device assemblies.

The containment chamber 108 of the cartridge 102 provides contact between collected fluid samples within the chamber and sensory apparatus, such as miniaturized electrodes and micro-sensors, for executing a series of calibration and diagnostic tests on the sample. The sensory apparatus (not shown) is distributed in and around the chamber 108 as known to those skilled in the art, and is electrically coupled to an exposed electrical contact 110 located within a port 112 along the containment chamber wall 114 of the cartridge 102. The exposed electrical contact 110 is contained within a mechanical coupler 116 as known to those skilled in the art, which is adapted to allow direct physical and electrical coupling of the cartridge 102 and a remote analytical device such as a hand-held analyzer, personal digital assistant (PDA) or VISOR®. Alternatively, the cartridge 102 may be coupled with a stand-alone computer workstation, or with any number of existing analyzers, such as those manufactured by the I-Stat Corporation of Princeton, N.J.

The electrical contact 110 of cartridge 102 allows the cartridge to engage an input port on a hand-held analytical device and electrically couple the sensory apparatus of the cartridge to a testing and analysis means within the analytical device. Once coupled to the cartridge 102, the analytical device gathers and processes information regarding the contained sample via the sensory apparatus within the containment chamber 108, and thereafter produces outputs which are displayed on an output mechanism, such as a liquid crystal display (LCD), analog display or light emitting diode (LED) indicator. Additional tests on the sample may be directed by activation of user interface mechanisms located on the analytical device. As the cartridge 102 is inexpensive and entirely disposable, upon completion of sample testing the cartridge is removed from the analytical device and disposed of.

With reference to the drawings of FIGS. 1, 2 and 3, the cartridge assembly 102 of the embodiment shown comprises a housing 118 having a top and bottom containment wall mechanically coupled to one another in a fashion creating a chamber 108 providing for the capture and containment of a sample substance such as blood, from a collection device 104. The housing 118 has a distal end 120 with a contaimnent chamber inlet orifice 122 formed therein, the orifice 122 being adapted to engage an outlet port 124 on the sample collection device 104.

As used herein, the term “proximal” refers to a location on the housing 118 closest to the person using the device and farthest from the patient in connection with which the device is used. Conversely, the term “distal” refers to a location on the housing 118 farthest from the person using the device and closest to the patient in connection with which the device is used.

The cartridge 102 may be removably coupled with a collection device 104 in a number of methods, such as through a press fit between the outlet port 124 of the collection device 104 and the inlet orifice 122 of the cartridge housing 118. The press fit mechanism of the collection device 104 removably holds the cartridge 102 firmly in place opposite the inlet port, or proximate end of the collection device 104, which may hold a collection element such as a needle assembly.

The press fit mechanism includes the outlet port 124 having a cylindrical extension 126 and an outlet passageway 128. The inlet orifice 122 of the housing 118 includes an opening 132, the inside diameter of which is slightly greater than the outside diameter of the cylindrical extension 126. To couple the cartridge 102 with the collection device 104, the collection device cylindrical extension 126 is inserted into the cylindrical opening 132 until the shoulder 130 is brought into contact with the distal end 120 of the housing 118, indicating a secure fit between collection device 104 and collection cartridge 102. Once the cartridge 102 is properly coupled with the collection device 104 as described above, an inlet passageway 134 creates a path between the containment chamber 108 and the outlet passageway 128 contained within the sample collection device 104. While engaged, a leak-proof seal is formed between the inlet passageway 134 and the outlet passageway 128, and a fluid sample is drawn into the containment chamber 108. The sample may be drawn into the containment chamber 108 in a number of ways, including vacuum created within the chamber 108 or capillary action of the inlet passageway 134.

The location of the press fit between collection device 104 and collection cartridge 102 at the proximal end of the collection device opposite the device needle allows easier and safer collection cartridge insertion and removal. All steps are performed opposite the sharp distal end of the collection device 104 preventing dangers to healthcare professionals who may accidentally or through negligent handling, stick themselves with needles. It will be noted that the embodiment above may use a number of press fit mechanisms to bring the inlet passageway 134 into contact with the outlet passageway 128 allowing sample collection.

Additionally, an adapter may be used at the outlet port 124 of the collection device 104 to allow the use of a wider range of collection cartridges. For example, collection cartridges by manufacturers such as the I-Stat Corporation, may be used in place of the collection cartridge 102 described above. The adapter may be used to engage any number of various collection cartridges and allow access between outlet passageway 128 of the collection device 104 and various inlet means employed by each collection cartridge manufacturer.

As known by those skilled in the art, a fluid sample may be easily collected by a sample collection device 104, where the collection device may be a syringe or lancet. The collected fluid sample within the device 104 is placed into contact with the outlet passageway 128 and is communicated to the proximal end of the collection device 104 through the natural capillary action of the passageway. The cartridge 102, once properly engaged with the collection device 104 as described above and shown in FIG. 2, allows the collection of a portion of the fluid sample from the outlet passageway 128 of the collection device 104 by the containment chamber 108 of the cartridge 102 via the inlet passageway 134. Once the cartridge 102 and the collection device 104 are properly engaged through the press fit mechanism described above, the outlet passageway 128 is brought into alignment and contact with the inlet passageway 134. The inlet passageway 134 creates a path between the containment chamber 108 and the outlet passageway 128 contained within the sample collection device 104. While engaged, a leak-proof seal is formed between the inlet passageway 134 and the outlet passageway 128 which allows the capillary action of the passageway 134 to communicate a fluid sample from the outlet passageway 128. The capillary action of the containment chamber 108 completes the capture of the fluid sample, which is then communicated throughout the chamber. Once a sufficient sample is captured and contained within the cartridge 102, the cartridge may be disengaged from the sample collection device 104 or, where practical to do so as described below, the cartridge may remain coupled to the collection device during testing. The exposed electrical contact 110 is located opposite the distal end 120 of the cartridge 102 thereby allowing the cartridge to remain engaged with the collection device 104 during sample testing with a hand-held analytical device as described below. Allowing the cartridge 102 and collection device 104 to remain coupled during testing may require shielding the collection device sharp distal end for safety, needle protection and other reasons obvious to those skilled in the art. Existing safety-engineered sharps protection systems include shields that pivot over needles, needles that retract into shields, and shields that move forward relative tot he needle, in order to contain the point of the needle. Needle pivoting, shielding or retracting systems and methods for collection devices are widely practiced and are described in numerous documents, such as U.S. Pat. No. 6,368,303 issued to Richard Caizza, and in U.S. Pat. No. 6,319,232 James Kashmer, the entire contents of each being incorporated herein by reference.

The cartridge assembly 102 further includes sensory apparatus which surrounds the collected sample within the containment chamber 108. As can be appreciated by one skilled in the art, the sensory apparatus may consist of a number of miniaturized electrodes and micro-sensors adapted to detect and measure various chemical and physical properties of the sample contained within the chamber 108. The sensory apparatus is distributed in and around the chamber 108 and inlet passageway 134 of the cartridge 102 and is electrically coupled to the exposed electrical contact 110 located externally along the containment chamber wall 114 opposite the distal end 120 of the cartridge 102. The exposed electrical contact 110 is contained within a mechanical coupler 116 adapted to allow direct physical and electrical coupling of the cartridge 102 and a like electrical contact and mechanical coupler located on a remote analytical device, such as a hand-held analyzer, personal digital assistant (PDA) or VISOR®, or a stand-alone computer workstation. Once within the containment chamber 108, the sample is subject to extensive analysis through direct and indirect contacts with the sensory apparatus, as directed by the remote analytical device.

Specifically, sample testing is achieved by engaging the cartridge 102 with the collection device 104 as described above. As noted earlier, an adapter may first be engaged with the collection device to accommodate alternate cartridges where necessary. Once engaged with the collection device 104, a sample is collected by the collection device into the cartridge 102. The filled cartridge 102 may then be removed from the sample collection device 104, or where practical to do so as described above, the cartridge may remain coupled to the collection device or adapter during testing. A remote analytical device 106 is then prepared to receive the filled cartridge 102 for testing. As known to those skilled in the art, many interface modules are provided to adapt hand-held devices to multiple uses, such as SPRINGBOARD® expansion modules for a personal digital assistant (PDA) or VISOR® as shown in FIG. 3. Where required, an interface module 134 is installed on the analytical device 106 which allows engagement with the exposed electrical contact 110 and mechanical coupler 116 of the cartridge 102. As appreciated by those skilled in the art, the analytical device 106 includes hardware and software adapted to access the sensory apparatus within the cartridge 102 and gather information on the sample contained therein, such as pH, pCO₂, pO₂ Na⁺, Ca⁺⁺, K⁺, hematocrit and glucose levels in the sample, in addition to sample temperature measurements. The analytical device may then perform numerous tests, configured by the user, on the sample contained within the containment chamber 108 of the cartridge 102 and display results via an output mechanism, such as a liquid crystal display (LCD), analog display or light emitting diode (LED) indicator on the analytical device. Additional tests or property detection may be directed by activation of user interface mechanisms located on the analytical device 106. Upon completion, the cartridge 102 and sample therein, are removed from the analytical device 106 and disposed. The cartridge 102 is fabricated as a sterile, disposable unit, preferably made of an inexpensive plastic, such as polyethylene, polypropylene, polyvinylidene chloride or the like. Additionally, sensory apparatus within the cartridge 102 are sufficiently inexpensive to allow single use and disposal.

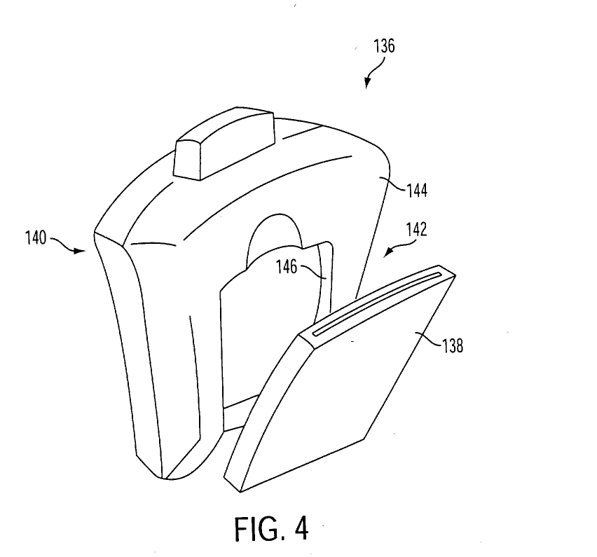

In another embodiment of the present invention, a diagram of an exemplary system 136 in accordance with an embodiment of the present invention is shown in FIG. 4. FIG. 4 is a view of a collection cartridge 138 disengaged from a collection device 140. The system 136 preferably includes a disposable cartridge 138 for providing a containment chamber into which fluid samples are placed for analysis, substantially the same as discussed above for the collection cartridge 102. The cartridge 138 is adapted to engage a collection device 140 which draws a fluid sample to the surface of a patient's skin for collection directly into the containment chamber of the cartridge 138.

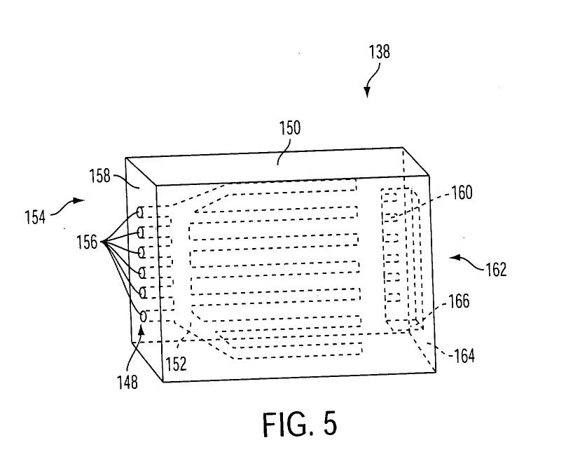

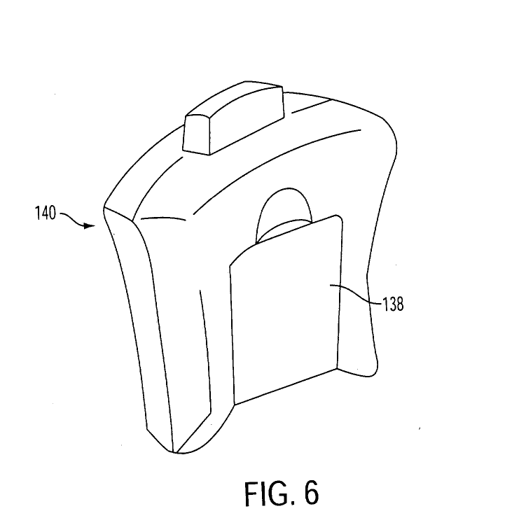

For the following discussion, reference will be made to all of FIGS. 4, 5 and 6, and as necessary, attention will be drawn to a particular figure. FIG. 5 is a view in partial section of the collection cartridge 138 disengaged from a collection device 140, and FIG. 6 is a view of the collection cartridge 138 engaged with a collection device 140 of FIG. 5, each according to an embodiment of the present invention.

With reference to the drawing of FIG. 4, a cartridge 138 may be mechanically coupled with a collection device 140, such as a lancet device, through a press fit mechanism 142 located at the distal end of the device body. In the embodiment shown in FIG. 4, the collection device 140 may be comprised of a handle housing 144, or a handle-lancet housing combination, including a press fit mechanism 142. The press fit mechanism 142 holds the cartridge 138 firmly in place within a recessed cartridge chamber 146 formed at the distal end of the collection device housing 144. The recessed cartridge chamber 146 is comprised of a locking slot or recess located within the handle housing 144 extending from the distal end of the housing to receive and secure the collection cartridge 138 to the collection device 140. The recessed cartridge chamber 146 is open at the distal end of the collection device, allowing the collection cartridge to position a containment chamber inlet orifice array 148 described below, adjacent to a collection device piercing mechanism also located at the distal end of the collection device 140 (not shown).

With reference to the drawings of FIGS. 4 and 5, the cartridge assembly 138 of the embodiment shown comprises a housing 150 having a top and bottom containment wall, mechanically coupled to one another in a fashion creating a containment chamber 152 providing for the capture and containment of a sample substance substantially the same as discussed above for the collection cartridge 102. The housing 150 has a distal end 154 with a containment chamber inlet orifice array 148 formed therein. The orifice array 148 includes one, or any number of inlet passageways 156 extending through the flat surface 158 of the housing 150 allowing access between the surface 158 and the containment chamber 152 within the cartridge 138.

The containment chamber 152 of the cartridge provides contact between collected fluid samples within the chamber and sensory apparatus for executing a series of calibration and diagnostic tests on the sample substantially the same as discussed above for the collection cartridge 102. The sensory apparatus (not shown) is distributed in and around the chamber 152 as known to those skilled in the art, and is electrically coupled to an exposed electrical contact 160 located within a port 162 along the containment chamber wall surface 164. The exposed electrical contact 160 is contained within a mechanical coupler 166 as known to those skilled in the art, which is adapted to allow direct physical and electrical coupling of the cartridge 138 and a remote analytical device such as a hand-held analyzer, personal digital assistant (PDA) or VISOR®, or stand-alone computer workstation, adapted to direct the testing of a sample within the chamber.

As known by those skilled in the art, the collection device 140, in this case a lancet, may be used to draw a blood sample from a patient. Additional details of lancet use are described in U.S. Pat. No. 4,677,979 issued to James A. Burns, the entire content of which is incorporated herein by reference. Once properly engaged with the collection device 140 as described above and shown in FIG. 6, the cartridge 138 allows the collection of a portion of the drawn fluid sample to the containment chamber 152 of the cartridge 138 via the inlet orifice array 148 using methods such as the capillary action of the inlet passageways 156 substantially as described with the first embodiment above. The fluid sample is placed into contact with the orifice array 148 and is communicated to the containment chamber 152 through the natural capillary action of the inlet passageways 156. Once the cartridge 138 and the collection device 140 are properly engaged through the press fit mechanism described above, the orifice array 148 is brought into position adjacent to the lancet mechanism allowing the inlet passageways 156 to create a path between the containment chamber 152 and any exposed fluid. While engaged, the capillary action of the inlet passageways 156 can communicate a fluid sample from the orifice array, and the capillary action of the containment chamber 152 completes the capture of the fluid sample, which is then communicated throughout the chamber. Other methods, such as a vacuum may also be used to draw the sample into the containment chamber 152.

Once a sufficient sample is captured and contained within the cartridge 138, the cartridge may be disengaged from the sample collection device 140 and placed in an analytical device for testing. Alternatively, the cartridge may remain engaged with the sample collection device and both may be placed in the analytical device for testing. Sample testing is achieved by removing the filled cartridge 138 from the sample collection device 140, and engaging the exposed electrical contact 160 and mechanical coupler 166 with a like electrical contact and mechanical coupler located on a remote analytical device substantially the same as discussed above for the collection cartridge 102. Sample testing may then be directed as described above.

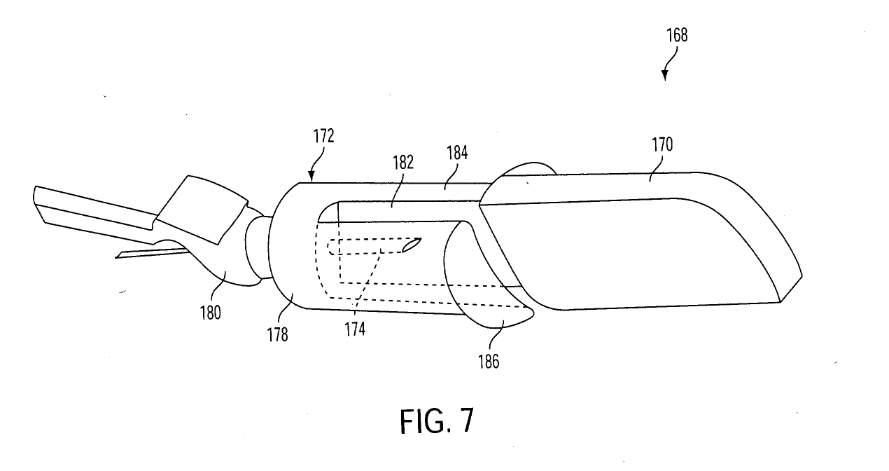

In another embodiment of the present invention, a diagram of an exemplary system 168 in accordance with an embodiment of the present invention is shown in FIG. 7. FIG. 7 is a view of a collection cartridge 170 disengaged from a collection device 172. The system 168 preferably includes a disposable cartridge 170 for providing a containment chamber into which fluid samples are placed for analysis, substantially the same as discussed above for the collection cartridge 102. The cartridge 170 is evacuated prior to use, and adapted to engage a collection device 172, used to collect a fluid sample from a patient and transfer the sample directly into the containment chamber of the cartridge 170.

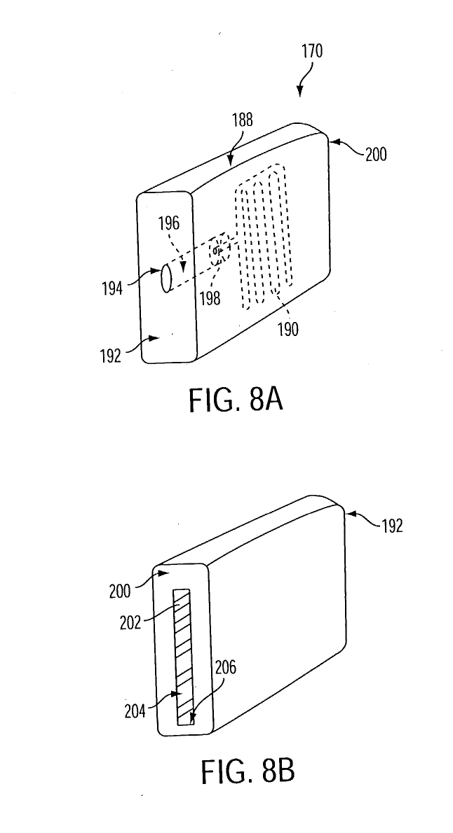

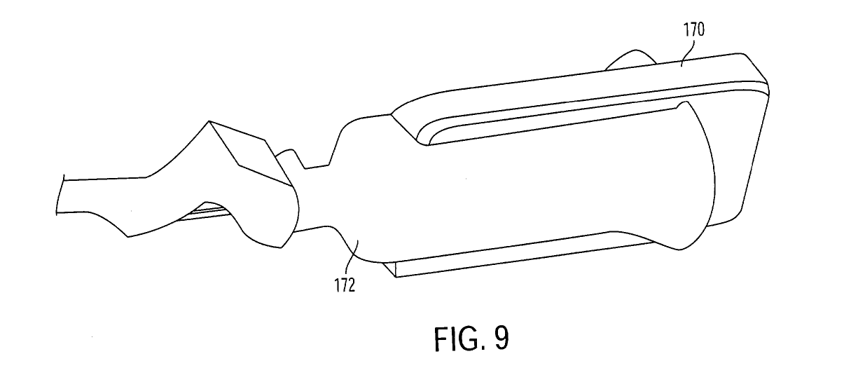

For the following discussion, reference will be made to all of FIGS. 7, 8 and 9, and as necessary, attention will be drawn to a particular figure. FIG. 7 is a view in partial section of an example of an evacuated removable cartridge disengaged from a collection device, and FIGS. 8A and 8B are views of the evacuated removable cartridge. FIG. 9 is a view of the evacuated removable cartridge of FIG. 7 engaged with the collection device according to an embodiment of the present invention

With reference to the drawing of FIG. 7, an evacuated cartridge 170 may be mechanically coupled with a collection device 172, such as a syringe device, through a press fit mechanism located at the proximal end of the handle housing 172. In the embodiment shown in FIG. 7, the collection device 172 may be comprised of a handle housing 178, including a press fit mechanism and a shielded piercing member 180 at a distal end. The press fit mechanism holds the cartridge 170 firmly in place within a recessed cartridge slot 182 formed at the proximal end of the collection device housing 178. The recessed cartridge slot 182 is comprised of first and second opposing tabs 184 and 186 extending as a partial housing wall about the circumference of the housing 178, where the radius of the partial housing wall is defined by the slot 182. The locking slot 182 is located between tabs 184 and 186 within the handle housing 178 and extends from the distal end of the housing to receive and secure the collection cartridge 170 to the collection device 172. A piercing member, such as an intravenous needle 174, in fluid communication with the shielded piercing member 180, is positioned within the slot 182, and is adapted to access the vacuum of the evacuated cartridge 170, allowing a transfer of the sample from the collection device 172 to the cartridge 170.

With reference to the drawings of FIGS. 7, 8A and 8B, the cartridge assembly 170 of the embodiment shown comprises a housing 188 having a top and bottom containment wall, mechanically coupled to one another in a fashion creating a containment chamber 190 providing for the capture and containment of a sample substance substantially the same as discussed above for the collection cartridge 102. The housing 188 has a distal end 192 with a containment chamber inlet orifice 194 formed therein. The orifice 194 includes an inlet passageway 196 extending from the distal end 192 and the containment chamber 190 within the cartridge 170, to mate with the intravenous needle 174 of the collection device.

In this embodiment, or any embodiment described above in which a vacuum is required within the containment chamber to provide the mechanism for transferring a fluid sample, the vacuum can be provided as part of the manufactured cartridge, or created prior to use through various cartridge construction techniques.

In the evacuated cartridge embodiment shown in FIG. 7, a vacuum can be created in the chamber 190 using a pliable cartridge body and a valve mechanism located at 198 to contain the vacuum within the chamber 190 until accessed by the intravenous needle 174 of the collection device 172 during engagement. The cartridge 170 can be manufactured having a contained vacuum for collection purposes, or, the cartridge can be manufactured having a pliable body for use with the valve mechanism for creating a contained vacuum in the field during use. The pliable body of the cartridge 170 can be manually compressed prior to use to evacuate the containment chamber 190 of the cartridge. The pliable body of the cartridge when released, returns to an original position, creating and capturing a vacuum within the containment chamber 190 via a valve mechanism located at 198. The valve mechanism 198 allows single direction air flow for the purpose of vacuum creation. The mechanism can be constructed of a material that is resilient, yet easily penetrable by a cannula, such as the intravenous needle 174.

The containment chamber 190 of the cartridge 170 provides contact between collected fluid samples within the chamber and sensory apparatus for executing a series of calibration and diagnostic tests on the sample substantially the same as discussed above for the collection cartridge 102. The sensory apparatus (not shown) is distributed in and around the chamber 190 as known to those skilled in the art, and is electrically coupled to an exposed electrical contact 202 located within a port 204 along the containment chamber wall surface 200. The exposed electrical contact 202 is contained within a mechanical coupler 206 as known to those skilled in the art, which is adapted to allow direct physical and electrical coupling of the cartridge 170 and a remote analytical device such as a hand-held analyzer, personal digital assistant (PDA) or VISOR®, or stand-alone computer workstation, adapted to direct the testing of a sample within the chamber.

As known by those skilled in the art, the collection device 172 may be used to draw a blood sample from a patient. Once properly engaged with the collection device 172 as described above and shown in FIG. 9, the cartridge 170 allows the collection of a portion of the drawn fluid sample to the containment chamber 190 of the cartridge 170 via the intravenous needle 174 of the collection device using methods such as capillary action or vacuum. The collected fluid sample within the collection device 172 is communicated to the proximal end of the collection device through the intravenous needle 174. Once the evacuated cartridge 170 and the collection device 172 are properly engaged through the press fit mechanism described above, the intravenous needle 174 accesses the inlet passageway 196 and valve mechanism 198, releasing the contained vacuum with in the evacuated cartridge 172. The inlet passageway 196 and valve mechanism 198 create a path between the containment chamber 190 and the intravenous needle 174, allowing the released vacuum to urge a fluid sample from the intravenous needle 174 into the containment chamber 190. While engaged, a leak-proof seal is formed between the intravenous needle 174 and the valve mechanism 198, which allows the vacuum of the evacuated cartridge 170 to communicate a fluid sample from the intravenous needle 174 throughout the chamber 190.

In another embodiment of the evacuated cartridge 170, the valve mechanism can be replaced with a sealed membrane or a penetrable cartridge body. The evacuated cartridge 170 is manufactured including a contained vacuum within the containment chamber 190, and requires no additional preparation steps prior to use. In this embodiment, the proper engagement through the press fit mechanism described above results in the intravenous needle 174 accessing the inlet passageway 196 and piercing the sealed membrane or cartridge body, and allowing fluid communication substantially as described above. Such embodiments of an evacuated cartridge based system therefore, also allows mating with existing sample access products available.

Once a sufficient sample is captured and contained within the cartridge 170, the cartridge may be disengaged from the sample collection device 172 and placed in an analytical device for testing. Sample testing is achieved by removing the filled cartridge 170 from the sample collection device, and engaging the exposed electrical contact 202 and mechanical coupler 206 with a like electrical contact and mechanical coupler located on a remote analytical device substantially the same as discussed above for the collection cartridge 102. Sample testing may then be directed as described above. Alternatively, the cartridge may remain engaged with the sample collection device and, after shielding the piercing member 180, both may be placed in the analytical device for testing.

The collection cartridge disclosed in each embodiment provides the ability to obtain immediate, reliable and accurate testing of fluid samples without the processing delays associated with traditional laboratories. Moreover, the cartridge greatly reduces the quantity of fluid sample required from the patient to perform these tests.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

What is claimed is:

1. A cartridge assembly for collecting fluid samples and performing fluid sample analysis, comprising:
a collection device adapted to collect fluid and having opposite ends with an inlet port formed at a distal end for communicating fluids into a first passageway of said device, said first passageway extending between said ends and accessible through an outlet port formed at a proximal end, said outlet port adapted to removably engage an inlet orifice of a collection cartridge;
said collection cartridge comprising a housing having said inlet orifice and a top and bottom containment wall, said top wall mechanically coupled to said bottom wall to define a containment chamber, wherein said containment chamber is accessible through said inlet orifice, said inlet orifice further adapted to removably engage said outlet port of said collection device and allow communication of a fluid sample from said first passageway of said collection device into said containment chamber of said collection cartridge, said collection cartridge further comprising a first electrical connector;
said first electrical connector comprising a first set of electrical contacts adapted to engage a second electrical connector located on an analytical device, said first set of electrical contacts electrically coupled to sensory apparatus; and
said sensory apparatus comprising sensors adapted to detect fluid sample properties, said sensory apparatus disposed within said collection cartridge and electrically coupled to said first set of electrical contacts and accessible by said analytical device.

2. A cartridge assembly for collecting fluid samples and performing fluid sample analysis as claimed in claim 1, wherein said inlet port of said collection device comprises a collection element engagement mechanism.

3. A cartridge assembly for collecting fluid samples and performing fluid sample analysis as claimed in claim 2, wherein said inlet port of said collection device comprises a conical luer fitting.

4. A cartridge assembly for collecting fluid samples and performing fluid sample analysis as claimed in claim 2, wherein said collection element is selected from the group consisting of an intra-venous needle, intra-arterial needle, venous catheter, arterial catheter, capillary tube, microneedle array and lancet.

5. A cartridge assembly for collecting fluid samples and performing fluid sample analysis as claimed in claim 2, wherein said collection device comprises a shielding mechanism adapted to shield said collection element.

6. A cartridge assembly for collecting fluid samples and performing fluid sample analysis as claimed in claim 1, wherein said inlet orifice comprises a second passageway accessing said containment chamber of said collection cartridge.

7. A cartridge assembly for collecting fluid samples and performing fluid sample analysis as claimed in claim 1, wherein said outlet port of said collection device comprises a press fit mechanism adapted to engage said cartridge and form a leak-proof seal between said first passageway of said collection device and said second passageway of said collection cartridge.

8. A cartridge assembly for collecting fluid samples and performing fluid sample analysis as claimed in claim 7, wherein said fluid sample is communicated from said first passageway of said collection device into said containment chamber using a capillary action of at least one of said first passageway, second passageway and containment chamber.

9. A cartridge assembly for collecting fluid samples and performing fluid sample analysis as claimed in claim 7, wherein said fluid sample is communicated from said first passageway of said collection device into said containment chamber using a vacuum created within said containment chamber.

10. A cartridge assembly for collecting fluid samples and performing fluid sample analysis as claimed in claim 1, wherein said second electrical connector comprises a second set of electrical contacts, said second electrical connector adapted to engage said first electrical connector of said collection cartridge and electrically couple said first and second set of electrical contacts.

11. A cartridge assembly for collecting fluid samples and performing fluid sample analysis as claimed in claim 1, wherein said first electrical connector is adapted to engage said second electrical connector while said inlet orifice of said collection cartridge is removably engaged with said outlet port of said collection device.

12. A cartridge assembly for collecting fluid samples and performing fluid sample analysis as claimed in claim 1, wherein said analytical device is adapted to perform user directed analytical tests on said fluid sample using said sensory apparatus via said first and second sets of electrical contacts.

13. A cartridge assembly for collecting fluid samples and performing fluid sample analysis as claimed in claim 1, wherein said fluid sample properties include at least one of a pH, pCO₂, pO₂, pCl, pNO₃, Na⁺, Ca⁺⁺, K⁺, hematocrit and glucose level in said sample.

14. A collection cartridge for collecting fluid samples and performing fluid sample analysis, comprising:
a cartridge housing having an inlet orifice and a top and bottom containment wall, said top wall mechanically coupled to said bottom wall to define a containment chamber, wherein said containment chamber is accessible through said inlet orifice, said housing further adapted to removably engage a collection device such that said inlet orifice allows communication of a fluid sample provided by said collection device to said containment chamber, said collection cartridge further comprising a first electrical connector;
said first electrical connector comprising a first set of electrical contacts adapted to engage a second electrical connector located on an analytical device, said first set of electrical contacts electrically coupled to sensory apparatus; and
said sensory apparatus comprising sensors adapted to detect fluid sample properties, said sensory apparatus disposed within said collection cartridge and electrically coupled to said first set of electrical contacts and accessible by said analytical device.

15. A collection cartridge for collecting fluid samples and performing fluid sample analysis as claimed in claim 14, wherein said inlet orifice comprises at least one passageway accessing said containment chamber of said cartridge housing.

16. A collection cartridge for collecting fluid samples and performing fluid sample analysis as claimed in claim 15, wherein said inlet orifice is adapted to form a leakproof seal between said collection device and said at least one passageway of said inlet orifice of said cartridge housing.

17. A collection cartridge for collecting fluid samples and performing fluid sample analysis as claimed in claim 14, wherein said fluid sample is communicated from said collection device into said containment chamber using a capillary action of at least one of said inlet orifice and containment chamber.

18. A collection cartridge for collecting fluid samples and performing fluid sample analysis as claimed in claim 14, wherein said fluid sample is communicated from said collection device into said containment chamber using a vacuum created within said containment chamber.

19. A collection cartridge for collecting fluid samples and performing fluid sample analysis as claimed in claim 14, wherein said second electrical connector comprises a second set of electrical contacts, said second electrical connector adapted to engage said first electrical connector of said collection cartridge and electrically couple said first and second set of electrical contacts.

20. A collection cartridge for collecting fluid samples and performing fluid sample analysis as claimed in claim 14, wherein said first electrical connector is adapted to engage said second electrical connector while said inlet orifice of said collection cartridge is removably engaged to said outlet port of said collection device.

21. A collection cartridge for collecting fluid samples and performing fluid sample analysis as claimed in claim 14, wherein said analytical device is adapted to perform user directed analytical tests on said fluid sample using said sensory apparatus via said first and second sets of electrical contacts.

22. A collection cartridge for collecting fluid samples and performing fluid sample analysis as claimed in claim 14, wherein said fluid sample properties include at least one of a pH, pCO₂, pO₂, pCl, pNO₃, Na⁺, Ca⁺⁺, K⁺, hematocrit and glucose level in said sample.

23. A collection device for collecting fluid samples into a test cartridge assembly, comprising:
a device housing adapted to collect fluid, said device housing having opposite proximate and distal ends with an inlet port formed at said distal end for communicating fluids into a first passageway of said device housing, said first passageway extending between said proximate and distal ends and accessible through an outlet port; and
an outlet port adapted to removably engage a collection cartridge such that said collection cartridge communicates a fluid sample from said first passageway of said collection device via an inlet orifice of said collection cartridge.

24. A collection device for collecting fluid samples into a test cartridge assembly as claimed in claim 23, wherein said inlet port of said collection device comprises a collection element engagement mechanism.

25. A collection device for collecting fluid samples into a test cartridge assembly as claimed in claim 24, wherein said inlet port of said collection device comprises a conical luer fitting.

26. A collection device for collecting fluid samples into a test cartridge assembly as claimed in claim 24, wherein said collection element is selected from the group consisting of an intra-venous needle, intra-arterial needle, venous catheter, arterial catheter, capillary tube, microneedle array and lancet.

27. A collection device for collecting fluid samples into a test cartridge assembly as claimed in claim 24, wherein said collection device comprises a shielding mechanism adapted to shield said collection element.

28. A collection device for collecting fluid samples into a test cartridge assembly as claimed in claim 23, wherein said outlet port comprises a press fit mechanism adapted to form a leakproof seal between said first passageway of said collection device and said inlet orifice of said collection cartridge.

29. A collection device for collecting fluid samples into a test cartridge assembly, comprising:
a device housing having opposite proximate and distal ends with a recessed port formed adjacent to said distal end for removably securing a collection cartridge, said housing further comprises a lancet mechanism; and
said lancet mechanism adapted to expose fluid from a patient such that said collection cartridge communicates a fluid sample from said exposed fluid into a containment chamber via an inlet orifice array of said collection cartridge.

30. A collection device for collecting fluid samples into a test cartridge assembly as claimed in claim 29, wherein said inlet orifice array comprises at least one passageway accessing said containment chamber of said collection cartridge.

31. A collection device for collecting fluid samples into a test cartridge assembly as claimed in claim 29, wherein said recessed port comprises a press fit mechanism adapted to removably secure said collection cartridge.

32. A collection device for collecting fluid samples into a test cartridge assembly as claimed in claim 29, wherein said fluid sample is communicated from said exposed fluid into said containment chamber of said collection cartridge using a capillary action of at least one of said inlet orifice array and containment chamber.

33. A collection cartridge for collecting fluid samples and performing fluid sample analysis, comprising:
a cartridge housing having an inlet orifice and a top and bottom containment wall, said top wall mechanically coupled to said bottom wall to define a containment chamber, wherein said containment chamber is accessible through said inlet orifice, said housing further adapted to removably engage a collection device such that said inlet orifice allows communication of a fluid sample provided by said collection device into said containment chamber, said collection cartridge further comprising a first electrical connector;
said first electrical connector comprising a first set of electrical contacts adapted to engage a second electrical connector located on an analytical device, said first set of electrical contacts electrically coupled to sensory apparatus; and
said sensory apparatus comprising sensors adapted to detect fluid sample properties, said sensory apparatus disposed within said collection cartridge and electrically coupled to said first set of electrical contacts and accessible by said analytical device.

34. A collection cartridge for collecting fluid samples and performing fluid sample analysis as claimed in claim 33, wherein said inlet orifice comprises at least one passageway accessing said containment chamber of said cartridge housing.

35. A collection cartridge for collecting fluid samples and performing fluid sample analysis as claimed in claim 33, wherein said inlet orifice is adapted to seal a vacuum within said containment chamber.

36. A collection cartridge for collecting fluid samples and performing fluid sample analysis as claimed in claim 33, wherein said inlet orifice is adapted to release a vacuum within said containment chamber when said housing is removably engaged with said collection device.

37. A collection cartridge for collecting fluid samples and performing fluid sample analysis as claimed in claim 36, wherein said release of said vacuum urges said communication of said fluid sample provided by said collection device into said containment chamber.

38. A collection cartridge for collecting fluid samples and performing fluid sample analysis as claimed in claim 35, wherein said inlet orifice is a valve mechanism adapted to seal said vacuum within said containment chamber and release said vacuum through said engagement with said collection device.

39. A collection cartridge for collecting fluid samples and performing fluid sample analysis as claimed in claim 38, wherein at least one of said top and bottom walls are pliable and is adapted to displace air within said containment chamber via said valve mechanism.

40. A collection cartridge for collecting fluid samples and performing fluid sample analysis as claimed in claim 38, wherein at least one of said top and bottom walls are pliable and is adapted to return to an original position after displacement and create said vacuum within said containment chamber via said valve mechanism.

41. A collection cartridge for collecting fluid samples and performing fluid sample analysis as claimed in claim 35, wherein said inlet orifice is a seal adapted to release said vacuum through said engagement with said collection device.
